(19) United States
(12) Patent Application Publication
(10) Pub. No.: US 2016/0038917 A1
(43) Pub. Date: Feb. 11, 2016

(54) HYDROGENATION CATALYSTS

(71) Applicant: BASF Corporation, Florham Park, NJ (US)

(72) Inventors: Deepak S. Thakur, Solon, OH (US); William J. Carrick, Beachwood, OH (US)

(21) Appl. No.: 14/822,291

(22) Filed: Aug. 10, 2015

Related U.S. Application Data
(60) Provisional application No. 62/035,755, filed on Aug. 11, 2014.
(60) Provisional application No. 62/050,534, filed on Sep. 15, 2014.

(51) Int. Cl. B01J 21/16; C07C 209/36; B01J 37/00; C07C 29/145; B01J 35/10; B01J 37/04

(52) U.S. Cl. CPC B01J 21/16; B01J 35/1038; B01J 37/04; B01J 37/0009; C07C 29/145; C07C 209/36

(57) ABSTRACT

Catalysts for hydrogenation comprise a catalytic material and an inorganic matrix component, wherein the catalytic material comprises: at least one metal component comprising a metal selected from the group consisting of copper, manganese, zinc, nickel, cobalt, and iron; and an alkali metal component or an alkaline earth metal component; wherein the inorganic matrix component based on at least a silica sol component and a clay material; wherein the catalytic material and the inorganic matrix component are processed together to form the catalyst; and wherein the catalyst has a mesopore volume in the range of 50-90 by weight % of an overall pore volume. Catalysts are effective for converting acetophenone to methylphenyl carbinol and/or for converting nitrobenzene to aniline.

### CROSS-REFERENCE TO RELATED APPLICATIONS

This application claims the benefit of U.S. Provisional Patent Application No. 62/035,755, filed on Aug. 11, 2014, and 62/050,534, filed on Sep. 15, 2014, both of which are incorporated herein by reference in their entirety, for any and all purposes.

### TECHNICAL FIELD

This invention relates to catalysts that are particularly useful as hydrogenation catalysts, and more particularly, catalysts that are useful for hydrogenating carbonyl compounds and nitro-compounds to form alcohols and amines, respectively. The invention also relates to a method of preparing these catalysts and to the use of the catalysts in hydrogenation reactions.

### BACKGROUND

Hydrogenation is a chemical reaction that involves the addition of hydrogen (H₂) and is used in large scale industrial processes or smaller scale laboratory procedures. Copper is a known catalyst for hydrogenation reactions. U.S. Pat. No. 6,049,008 (Roberts), for example, is directed to chromium-free copper catalysts. U.S. Pat. No. 5,124,295 (Nebesh), for example, is directed to copper chromite catalysts. An exemplary carbonyl is a ketone, such as acetophenone, which can be hydrogenated to form an industrially useful feedstock, methylphenyl carbinol, according to hydrogenation reaction (1).

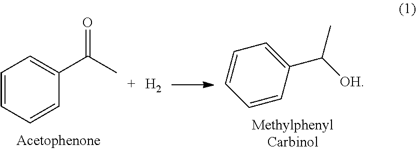

Many current commercial processes operate at high pressures, for example, in the range of 75-80 bar, to convert acetophenone to methyphenyl carbinol. For lower operating costs and increased safety measures, there is a desire to operate such processes at lower pressures in fixed bed reactors. At lower pressures, there is a need to provide catalysts that show at least the same, if not better, activity and selectivity that was achieved at the higher pressures. Undesired reactions, one of which is shown in reaction (2) for example, at these lower pressures (e.g., 25 bar) result in by-products that cause fouling of the catalyst and reactor. Reaction (2) shows dehydration of the alcohol to an olefin followed by hydrogenation to a hydrocarbon.

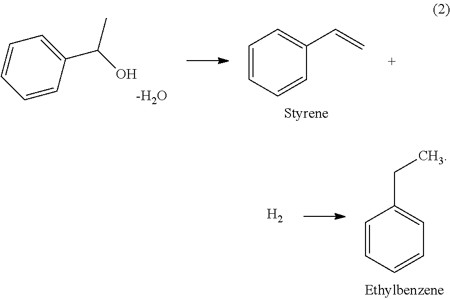

An exemplary nitro-compound, nitrobenzene, can be hydrogenated to form an industrially useful feedstock, aniline, according to hydrogenation reaction (3).

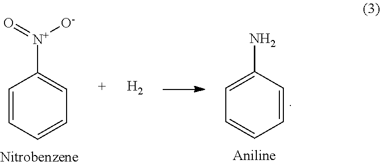 [SMILES: O=[N+]([O-])c1ccccc1 ; "H2" ; Nc1ccccc1]

Many current commercial processes operate at 180-220° C. and ambient pressure to convert nitrobenzene to aniline in fixed beds under vapor-phase. There is a need to provide catalysts that show at least the same, if not better, activity and selectivity. Undesired reactions (reaction 4) result in by-products that cause further hydrogenation. Reaction (4) shows hydrogenation of the aniline to an undesired amine.

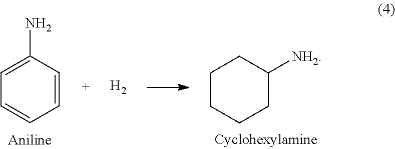 [SMILES: Nc1ccccc1 ; "H2" ; NC1CCCCC1]

There is a continuing need to provide catalysts that maximize desired hydrogenation products while eliminating by-product formation. It is also desirable to provide hydrogenation catalysts, methods for their manufacture and methods of use, which exhibit higher catalytic activity than existing catalysts.

### SUMMARY

Provided are catalysts for hydrogenation and methods of making and using the same. In a first aspect, a catalyst for hydrogenation comprises a catalytic material and an inorganic matrix component, wherein the catalytic material comprises: at least one metal component comprising: (a) a metal selected from the group consisting of copper, manganese, zinc, nickel, cobalt, and iron; (b) an alkali metal component and (c) optionally an alkaline earth metal component; wherein the inorganic matrix component is based on at least a silica sol component and a clay material; wherein the catalytic material and the inorganic matrix component are processed together to form the catalyst; and wherein the catalyst has a mesopore volume in the range of 50-90 by weight % of an overall pore volume.

In a specific aspect, a catalyst for hydrogenation is formed from a blend consisting essentially of copper oxide, sodium hydroxide, silica sol, and a clay component, which are processed together to form a catalyst that has a mesopore volume in the range of 50-90 by weight % of an overall pore volume.

Another aspect provides a method of making a catalyst for hydrogenation, the method comprising: mixing at least one metal component comprising a metal selected from the group consisting of copper, manganese, zinc, nickel, cobalt, and iron; and an inorganic matrix component based on at least a silica sol component and a clay material to form a dry mixture; adding a solution containing an alkali metal component to the dry mixture to form a blend; and forming the catalyst which has a mesopore volume in the range of 50-90 by weight % of an overall pore volume.

In a further aspect, provided are methods for making alcohols or amines comprising: providing a feedstock comprising a carbonyl compound or a nitro-compound; contacting the feedstock with any of the catalysts disclosed herein; and yielding alcohols or amines, respectively. Thus, any of the catalysts disclosed herein may be used for converting acetophenone to methylphenyl carbinol and/or for converting nitrobenzene to aniline.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

Provided are hydrogenation catalysts that are useful for hydrogenating carbonyl compounds and nitro-compounds to form alcohols and amines, respectively. Exemplary carbonyl compounds are ketones and aldehydes. Methods of making and using the same are also provided. These catalysts are formed from a catalytic material and an inorganic matrix component, which are processed together, for example, by extrusion or by tableting, to form the catalyst. The catalytic material comprises at least one metal component comprising a metal selected from the group consisting of copper, nickel, manganese, zinc, and cobalt in combination with an alkali metal component and optionally an alkaline earth metal component. The inorganic matrix component is formed from at least a silica sol and a clay material. Without intending to be bound by theory, the use of an alkali metal component results in a catalyst having excellent selectivity and activity for hydrogenation. Further delivering the alkali metal component separately from the silica component, for example sodium hydroxide and silica sol, respectively, rather than using an alkali silicate such as sodium silicate, results in a catalyst having a content of mesopores that facilitates the hydrogenation reactions and extends catalyst life.

Catalysts disclosed herein in extruded form show improved crush strength as compared to extruded catalysts formed using sodium silicate as a single source of both the alkali metal and the silica.

Specifically, these catalysts contain a significant amount of mesoporosity. Reference to “mesoporosity” or “mesopore” means those pores having a pore diameter in the range of 20 to 700 Angstroms (Å). That is, the pore volume of pores having a diameter in the range of 20 to 700 Å is in the range of 50%-90% by weight of the total pore volume, or 75%-86%, or even 80 to 85%. The catalyst may have a mesopore volume in the range of 0.21 to 0.33 cc/g, or even 0.30 to 0.32 cc/g and an overall pore volume in the range of 0.28 to 0.40 cc/g, or even 0.35 to 0.37 cc/g. The catalyst may have a surface area in the range of 20 to 90 m²/g.

Reference to a metal component means a material used to deliver a metal, for example metal oxides, which may be in solid or granular form. Thus, copper, manganese, zinc, nickel, cobalt, and/or iron may be delivered by their respective oxides.

Reference an “alkali metal component” or an “alkaline earth metal component” means a material used to deliver an alkali metal or an alkaline earth metal, for example metal hydroxides or carbonates, which may be in powder form or in an aqueous solution.

Reference to “inorganic matrix component” means a material suitable for binding components together to form a catalyst in a shape. Generally, the inorganic matrix component is extrudable and used to form extruded catalysts and/or the inorganic matrix component is able to form tableted catalysts. Thus, the inorganic matrix component, or binder material, may include silica, zinc oxide, zirconium oxide, clay such as Bentonite, silicates such as calcium silicate, etc., and mixtures thereof. In a preferred embodiment, the silica source is silica sol. Suitable clays include Attapulgite.

All references to pore diameters and pore volumes in the specification and claims of this application are based upon measurements utilizing mercury porosimetry. A typical method is described by R. Anderson, Experimental Methods in Catalytic Research, Academic Press, New York, 1968. The pore volumes are determined utilizing the catalysts in their oxide forms. That is, the pore diameters and pore volumes reported herein are obtained for the catalyst after calcination, but prior to any reduction of the oxide. Those skilled in the art often refer to the catalyst containing the metal oxides as the “oxide” or “oxide precursor” form of the catalyst.

It has been found that acidity from a feed and/or a catalyst surface can catalyze the undesirable side-reactions (2) and (4) under conditions vapor phase, fixed bed hydrogenation conditions. Without intending to be bound by theory, it is thought that the presence of the alkali metal component reduces acid sites on the catalyst surface, thereby, discouraging the undesirable side-reactions (2) and (4) under these conditions. Thus, the presence of an alkali metal, such as sodium, permits a higher selectivity for desired hydrogenation products as compared to catalysts without the alkali metal. These catalysts are also beneficial for long life in commercial low pressure operations, where selectivity for the hydrogenation products remains high even with periodic increases of temperature as the catalyst ages.

The metal present in the catalyst may be present as the reduced metal or oxide forms or as precursors to the reduced metal or oxide forms such as carbonates or nitrates which can be readily converted to the reduced metal or oxide forms or mixtures of two or more of any of these. The metals useful for the purposes may be present in one or more oxidation states. This invention also contemplates mixtures of two or more of these metals. Typically, the metal will be copper. Usually the catalyst has a total metal content of copper, manganese, zinc, nickel, cobalt, and iron of at least about 30%; typically from about 30% up to 85% by weight; preferably from about 35 up to 85% by weight, or even 55% to 85% by weight.

The catalyst also contains one or more promoter metals such as alkali or alkaline earth metals that are typically present in amounts from about 1% by weight up to about 10% by weight of the catalyst; preferably 0.5% by weight up to about 5% by weight. These metals may be present in the reduced metal or oxide forms or as precursors to such forms and in one or more oxidation states as discussed above. In one embodiment, the alkali metal component is an alkali metal hydroxide or carbonate where the alkali metal is selected from the group consisting of sodium (Na), potassium (K), rubidium (Rb), cesium (Cs), and combinations thereof. In another embodiment, the alkaline earth metal is selected from the group consisting of magnesium (Mg), calcium (Ca), strontium (Sr), barium (Ba), and combinations thereof.

Catalysts will generally not contain ingredients that will affect selectivity or acidity. For example, the catalysts will not contain zeolites, which would increase acidity. The compositions are also preferably free of added alumina, i.e., alumina other than that contributed by the clay incorporated in the composition as contemplated by the invention, which can also add to acidity. In addition, compositions are usually free of chromium in order to reduce exposure to such material. As used herein the catalyst is free of such materials if their presence is in an amount that does not materially affect the physical, chemical and catalytic characteristics of the compositions when compared to those which are completely free of such materials. Preferably, if present, such materials will be present in trace amounts, but in amounts not greater than about 1.5% by weight, more preferably not greater than 0.5% weight.

The silica component of the compositions can be from natural or synthetic sources, or preferably, is formed in situ (hereinafter “in situ”) during the preparation of the shaped catalyst composition. Preferably, the silica sources are clay and silica sol. The silica particle size in the silica sol may be in the range of 10-100 nm. A preferred silica sol is sold under the trade name Nalco 1034A having a typical particle size of 20 nm, a surface area of 150 m²/g, and 34% silica (as SiO₂). Typically, the catalyst composition contains up to about 50 wt.-% silica; usually, from about 10% up to about 40 wt. %; and preferably, from about 20% up to about 35% by weight.

The catalytic material also contains one or more clay materials. The clays suitable for use in this invention include alumino-silicate clays such as attapulgites, sepiolites, serpentines, kaolinites, calcium montmorillonites and mixtures thereof. Clays useful in making compositions of the instant invention include those obtained from the Meigs—Attapulgus—Quincy fullers earth districts, located in southwest Georgia and northern Florida.

For purposes herein, the term “attapulgite” is used to mean chain lattice type clay minerals, encompassing minerals and mineral groups variously referred to in the literature as “attapulgite,” “palygorskite,” “sepiolite,” and “hormite.” Typically, the clays suitable for use in the instant invention contain a major amount of attapulgite. As used herein, “major amount” shall mean and refer to a component which is present in the largest amount of any of the components present.

Those skilled in the art will be familiar with methods to determine the relative amounts of various mineral phases present in such clays. The clays suitable for use in the practice may be undried, dried or calcined. The free moisture content of the clays suitable for use in this invention is preferably from about 3 up to about 8 weight percent. As used herein, the “free-moisture content” is the amount of water removed from the clay by heating to constant weight at 100° C. (220° F.). Typically, the clay material as mined contains up to about 45% by weight free moisture content.

The clay material for use in this invention is preferably powdered and typically has particles having mesh sizes of less than about 200 mesh (U.S. Standard), preferably less than about 325. The composition may contain up to about 30% by weight of at least one clay material; typically from about 1% up to about 30% by weight; preferably from about 3 up to about 15% by weight.

Catalysts can be provided as tablets or extrudates. One way to process the blend of all of the ingredients is to extrude it through a shaping orifice to form an extruded catalyst body, or extrudate. Other catalyst bodies can be shaped into spheres or any other convenient formation. Another way is to tablet the catalysts.

Before describing several exemplary embodiments of the invention, it is to be understood that the invention is not limited to the details of construction or process steps set forth in the description. The invention is capable of other embodiments and of being practiced or being carried out in various ways. In the following, preferred designs for the catalysts are provided, including such combinations as recited used alone or in unlimited combinations, the uses for which include industrial hydrogenation processes including, but not limited to, converting acetophenone to methylphenyl carbinol and/or for converting nitrobenzene to aniline.

In embodiment 1, a catalyst for hydrogenation comprises a catalytic material and an inorganic matrix component, wherein the catalytic material comprises: at least one metal component comprising: (a) a metal selected from the group consisting of copper, manganese, zinc, nickel, cobalt, and iron; (b) an alkali metal component; and (c) optionally an alkaline earth metal component; wherein the inorganic matrix component is based on at least a silica sol component and a clay material; wherein the catalytic material and the inorganic matrix component are processed together to form the catalyst; and wherein the catalyst has a mesopore volume in the range of 50-90 by weight % of an overall pore volume.

Embodiment 2 is specifically a catalyst for hydrogenation that is formed from a blend consisting essentially of copper oxide, sodium hydroxide, silica sol, and a clay component, which are processed together to form a catalyst that has a mesopore volume in the range of 50-90 by weight % of an overall pore volume.

Embodiment 3 is a method of making a catalyst for hydrogenation, the method comprising: mixing at least one metal component comprising a metal selected from the group consisting of copper, manganese, zinc, nickel, cobalt, and iron; and an inorganic matrix component based on at least a silica sol component and a clay material to form a dry mixture; adding a solution containing an alkali metal component to the dry mixture to form a blend; and forming the catalyst which has a mesopore volume in the range of 50-90 by weight % of an overall pore volume.

Embodiments 1, 2, or 3 may have one or more of the following design features:

the metal comprises copper and that is prepared from a blend of: an amount of the copper component in the range of 30 to 85% by weight of the blend; an amount of the alkali metal component in the range of 0.5 to 5.0% by weight of the blend; and a combined amount of the silica sol and clay material in the range of 15 to 70% by weight of the blend (or even 15 to 40% by weight);

the alkali metal component is an alkali metal hydroxide or carbonate where the alkali metal is selected from the group consisting of sodium (Na), potassium (K), rubidium (Rb), cesium (Cs), and combinations thereof;

the alkali earth metal component is present in the range of 0.5 to 5.0% by weight of the blend and selected from the group consisting of magnesium (Mg), calcium (Ca), strontium (Sr), barium (Ba), and combinations thereof.

the catalyst has a mesopore volume in the range of 0.21 to 0.33 cc/g and an overall pore volume in the range of 0.28 to 0.40 cc/g;

the catalyst has a surface area in the range of 20-90 m²/g or even 25 to 70 m²/g;

the catalyst has an increased hydrogenation activity as compared to a copper silicate catalyst having no alkali metal component or alkaline earth metal component as used in hydrogenation reactions;

the clay material comprises an attapulgite, a sepiolite, a serpentine, a kaolinite, a calcium montmorillonite, or mixtures thereof;

the silica has a particle size in the range of 10-100 nm;

the catalyst in extruded form having a mesopore volume in the range of 0.29 to 0.33 cc/g and an overall pore volume in the range of 0.35 to 0.40 cc/g;

the catalyst in extruded form having a crush strength of 2 pounds per mm or more;

the catalyst in extruded form having a crush strength in the range of 4-5 pounds per mm; and

the catalyst in tablet form having a mesopore volume in the range of 0.21 to 0.25 cc/g and an overall pore volume in the range of 0.28 to 0.31 cc/g.

Embodiment 4 is a method for making alcohols or amines comprising: providing a feedstock comprising a carbonyl compound or a nitro-compound; contacting the feedstock with any of the catalysts of Embodiments 1 or 2 and any combination of design features disclosed herein; and yielding alcohols or amines, respectively. Thus, any of the catalysts of Embodiments 1 or 2 and any combination of design features disclosed herein may be used for converting acetophenone to methylphenyl carbinol and/or for converting nitrobenzene to aniline. The catalyst may be effective to convert 80% or more of acetophenone to methylphenyl carbinol under continuous stirred tank reactor (CSTR) conditions at 20.7 bar and feed rate of 150 cc-hr⁻¹ with 33 cc catalyst and temperatures up to 100° C. at steady state. The catalyst may be effective to maintain 90% or more selectivity of acetophenone to methylphenyl carbinol for at least 250 hours. The catalyst may also be effective to maintain 97% or more selectivity of nitrobenzene to aniline under fixed bed conditions at 220° C. and 0.3 LHSV hr^{31 1} for at least 250 hours.

The present invention, thus generally described, will be understood more readily by reference to the following examples, which are provided by way of illustration and are not intended to be limiting of the present invention.

### EXAMPLES

Example 1

A series of chromium-free, copper silicate catalysts having varying levels of copper oxide, sodium, and surface area were prepared as follows. These catalysts had mesopore volumes in the range of about 77 to 86% of the entire pore volume. Copper oxide, clay, calcium hydroxide (lime), alkali metal source (for Examples 1B and 1C only, sodium hydroxide solution), and silica sol were mixed and kneaded. The mixture was then extruded with an extruder and dried at a temperature range of 120-150° C. The extrudates were then calcined at 500-700° C. to a desired surface area. The catalysts had the following properties, where “3F” means 3-fluted or tri-lobe:

TABLE 1

| | Example | Example | Example |
|---|---|---|---|
| | 1A | 1B | 1C |
| % Na₂O @ 500° C. | 0 | 0.5 | 3.3 |
| Size/shape | 1/16″ 3F | 1/16″ 3F | 1/16″ 3F |
| Surface area (m²/g) | 36 | 30 | 38 |
| Hg Pore Volume (cc/g) | | | |
| up to 30 Å | 0.00 | 0.00 | 0.00 |
| up to 90 Å | 0.015 | 0.015 | 0.013 |
| up to 120 Å | 0.03 | 0.025 | 0.026 |
| up to 600 Å | 0.31 | 0.26 | 0.30 |
| up to 700 Å | 0.32 | 0.30 | 0.30 |
| up to 95000 Å | 0.37 | 0.39 | 0.37 |
| Avg. Pore Diameter (Å) | 260 | 300 | 264 |
| Packed density (g/cc) | 0.81 | 0.90 | 0.9 |
| Wt % mesopore volume | 86 | 77 | 81 |
| of total volume | | | |
| Crush strength, lbs./mm | 4.6 | 4.2 | 4.8 |
| % CuO @ 500° C. | 64 | 75 | 74 |
| % SiO₂ @ 500° C. | 21 | 17 | 15 |
| % CaO @ 500° C. | 14 | 5 | 6 |
| % Al₂O₃ | 1 | 1 | 0.65 |

Example 2

### Comparative

A chromium-free, copper silicate catalyst having lower surface area and lower mesopore volume as compared to the catalysts of Example 1 was prepared as follows. The copper oxide, clay, calcium lime, alkali metal source (sodium silicate) were mixed and kneaded. The mixture was then extruded with an extruder and dried at a temperature range of 120-150° C. The extrudates were then calcined at 500-600° C. to a desired surface area. The catalyst had the following properties:

TABLE 2

| | Example 2 |
|---|---|
| % Na₂O @ 500° C. | 3.3 |
| Size/shape | 1/16″ 3F |
| Surface area (m²/g) | 15 |
| Hg Pore Volume (cc/g) | |
| up to 30 Å | 0.00 |
| up to 90 Å | 0.003 |
| up to 120 Å | 0.005 |
| up to 600 Å | 0.124 |
| up to 700 Å | 0.150 |
| up to 95000 Å | 0.340 |
| Avg. Pore Diameter (Å) | 575 |
| Packed density (g/cc) | 1.05 |
| Wt % mesopore volume | 44 |
| of total volume | |
| Crush strength, lbs./mm | 1.8 |
| % CuO @ 500° C. | 75 |
| % SiO₂ @ 500° C. | 14 |
| % CaO @ 500° C. | 5.0 |
| % Al₂O₃ | 0.5 |

Example 3

### Testing

The chromium-free, copper silicate catalyst catalysts of Examples 1 and 2 were tested in a 1-liter Continuously Stirred Tank Reactor (CSTR) with 33 cc of catalyst placed in a basket. Activity and acetophenone conversion were measured under conditions of pressure 20.7 bar (300 psi), temperature 100° C., feed flow rate 150 cc-hr⁻¹, hydrogen flow rate 50.8 Liters hr⁻¹. The catalysts yielded the following conversions and selectivities.

TABLE 3

| | Example | Example | Example | Comparative |
|---|---|---|---|---|
| | 1A | 1B | 1C | Example 2 |
| % Na₂O @ | 0 | 0.5 | 3.3 | 3.3 |
| 500° C. | | | | |

% acetophenone conversion

| | | | | |
|---|---|---|---|---|
| T 10 hrs | 62 | 87 | 84.9 | — |
| T 25 hrs | 55 | 84.4 | 85.2 | 53.6 |
| T 50 hrs | — | 79.5 | 89.3 | 53.2 |
| T 100 hrs | — | 74.1 | 86.1 | 51.2 |
| T 150 hrs | — | 72.2 | 90.0 | 49.8 |
| T 200 hrs | — | 67.3 | 91.6 | 48.0 |
| T 250 hrs | — | 61.5 | 86.9 | 48.0 |
| T 300 hrs | — | 60 | 87.2 | — |

% Methyl Benzyl Alcohol Selectivity

| | | | | |
|---|---|---|---|---|
| T 25 hrs | 64.3 | 84.9 | 85.19 | 53.7 |
| T 50 hrs | 62.4 | 83 | 97.7 | 53.1 |
| T 100 hrs | 56.0 | 79.5 | 86.2 | 49.8 |
| T 150 hrs | 55.4 | 80 | 90.1 | 51.9 |
| T 200 hrs | — | 77 | 91.6 | 49.8 |
| T 250 hrs | — | 72.2 | 88 | 48.1 |
| T 300 hrs | — | — | 92.5 | — |

The data of Table 3 show that Examples 1B and 1C, which used individual sources of sodium (in the form of sodium hydroxide) and silica (in the form of silica sol) showed higher surface area and crush strength as compared to Comparative Example 2. Inclusion of sodium as shown in Examples 1B and 1C improves selectivity and catalyst life as compared to Example 1A (without sodium). Use of silica sol as shown in Examples 1A, 1B, and 1C offers higher surface area and higher volume of mesopores as compared to Comparative Example 2, which improves activity and conversion.

Example 4

A sodium-containing, chromium-free, copper silicate catalyst was prepared as follows. This catalyst had a mesopore volume that was 75% of the entire pore volume. Copper carbonate, clay, calcium lime, alkali metal source (sodium hydroxide solution), water and silica sol were mixed and kneaded. The mixture was then dried at a temperature range of 100-125° C. Dried pill-mix was granulated; formed into 3/16″ tablets and then calcined at 500-700° C. to a desired surface area. The catalyst had the following properties:

TABLE 4

| | Example 4 |
|---|---|
| % Na₂O @ 500° C. | 3.0 |
| Size/shape | 3/16″ cylinder |
| Surface area (m²/g) | 27 |
| Hg Pore Volume (cc/g) | |
| up to 30 Å | 0.05 |
| up to 90 Å | 0.06 |
| up to 120 Å | 0.11 |
| up to 600 Å | 0.20 |
| up to 700 Å | 0.21 |
| up to 95000 Å | 0.28 |
| Packed density (g/cc) | 1.3 |
| Wt % mesopore volume | 75 |
| of total volume | |
| Crush strength, lbs | 20 |
| % CuO @ 500° C. | 56 |
| % SiO₂ @ 500° C. | 20 |
| % CaO @ 500° C. | 18 |
| % Al₂O₃ | 1 |

Example 5

### Comparative

A chromium-free, copper silicate catalyst without sodium and using a colloidal silica source was prepared as follows. The copper precursor in the form of cupric oxide, clay, calcium hydroxide, water and colloidal silica were mixed. The final mixture was dried at a temperature range of 120-150° C. Dried pill-mix was granulated; formed into 3/16″ tablets and then calcined at 500-700° C. to a desired surface area. The catalyst had the following properties:

TABLE 5

| | Example 5 |
|---|---|
| % Na₂O @ 500° C. | 0 |
| Size/shape | 3/16″, cylinder |
| Surface area (m²/g) | 40 |
| Hg Pore Volume (cc/g) | |
| up to 30 Å | 0.01 |
| up to 90 Å | 0.04 |
| up to 120 Å | 0.06 |
| up to 600 Å | 0.24 |
| up to 700 Å | 0.25 |
| up to 95000 Å | 0.29 |
| Packed density (g/cc) | 1.2 |
| Crush strength, lbs | 20 |
| % CuO @ 500° C. | 60 |
| % SiO₂ @ 500° C. | 20 |
| % CaO @ 500° C. | 18 |
| % Al₂O₃ | 1 |

### Example 6

### Testing

The chromium-free, copper silicate catalysts of Examples 4 and 5 were tested for aniline selectivity at 100% nitrobenzene conversion versus temperature under conditions of LHSV 0.3 hr⁻¹ and hydrogen:nitrobenzene 10:1. The catalyst yielded the following selectivities, where steady state was achieved at each temperature.

TABLE 6a

| | | Comparative |
|---|---|---|
| | Example 4 | Example 5 |
| % Na₂O @ 500° C. | 3 | 0 |

% Aniline Selectivity

| | | |
|---|---|---|
| 200° C. | 99.8 | 98.6 |
| 220° C. | 99.5 | 97.9 |
| 240° C. | 97.8 | 91.3 |

The data of Table 6 show that Example 4, which used individual sources of sodium (in the form of sodium hydroxide) and silica (in the form of silica sol) showed better aniline selectivity as compared to Comparative Example 5, which did not have any sodium. Specifically, Example 4 was able to maintain more than 97% selectivity over time as the temperature was increased.

The catalysts of Examples 4 and 5 were also tested for acidity, measurements for which were taken using Diffuse Reflectance Fourier-Transform infrared spectrometry on a Perkin-Elmer PC 1000 IR spectrometer. The powders were hand ground and analyzed in-situ using a Spectra-Tech diffuse reflectance high temperature camber. The samples were then dehydrated at 450° C. under flowing N₂ and then allowed to cool to room temperature prior to probing with pyridine. Data was collected after 40° C. desorption and reported as μmoles/gram after smoothing and deconvolution.

TABLE 6b
Pyridine-IR Acidity Measurements
of Cu/SiO₂ tablets - μmole/gram

| | | Comparative |
|---|---|---|
| | Example 4 | Example 5 |
| Brönsted 40° C. | — | 1 |
| Lewis 40° C. | 32 | 62 |

The data of Table 6b shows that the catalyst of Example 4 exhibited less acidity than the catalyst of Comparative Example 5.

Example 7

### Comparative

A chromium-free, copper silicate catalyst formed without sodium and using a combined source of alkali and silica, specifically sodium silicate, was prepared as follows. The copper precursor in the form of cupric oxide, clay, calcium hydroxide, water and sodium silicate were mixed. The final mixture was dried at a temperature range of 120-150° C. Dried pill-mix was granulated; formed into 3/16″ tablets and then calcined at 400-700° C. The catalyst had the following properties:

TABLE 7

| | Comparative |
|---|---|
| | Example 7 |
| % Na₂O @ 500° C. | 0 |
| Size/shape | 3/16″, cylinder |
| Hg Pore Volume (cc/g) | |
| up to 60 Å | 0.017 |
| up to 90 Å | 0.016 |
| up to 120 Å | 0.016 |
| up to 600 Å | 0.042 |
| up to 700 Å | 0.049 |
| up to 94700 Å | 0.145 |
| Wt % mesopore volume | 34 |
| of total volume | |

Table 7 shows that the mesopore volume of Comparative Example 7 (34 wt-%) is significantly lower than that of Example 4 (75 wt-%).

While certain embodiments have been illustrated and described, it should be understood that changes and modifications can be made therein in accordance with ordinary skill in the art without departing from the technology in its broader aspects as defined in the following claims.

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

What is claimed is:

1. A catalyst for hydrogenation comprising a catalytic material and an inorganic matrix component, wherein the catalytic material comprises:
a metal component comprising a metal selected from the group consisting of copper, manganese, zinc, nickel, cobalt, and iron; and
an alkali metal component;
wherein:
the inorganic matrix component is based on at least a silica sol component and a clay material;
the catalytic material and the inorganic matrix component are processed together to form the catalyst; and
the catalyst has a mesopore volume in the range of 50-90 weight % of an overall pore volume.

2. The catalyst of claim 1, wherein the catalytic material further comprises an alkaline earth metal component.

3. The catalyst of claim 2, wherein the metal component comprises copper and is prepared from a blend of:
an amount of the copper component in the range of 30 to 85% by weight of the blend;
an amount of the alkali metal component in the range of 0.5 to 5.0% by weight of the blend; and
a combined amount of the silica sol and clay material in the range of 15 to 70% by weight of the blend.

4. The catalyst of claim 2 further comprising the alkali metal component which is an alkali metal hydroxide or carbonate where the alkali metal is selected from the group consisting of sodium (Na), potassium (K), rubidium (Rb), cesium (Cs), and a combination of any two or more thereof.

5. The catalyst of claim 2 further comprising an alkali earth metal component selected from the group consisting of magnesium (Mg), calcium (Ca), strontium (Sr), barium (Ba), and a combination of any two or more thereof.

6. The catalyst of claim 1 having a mesopore volume in the range of 0.21 to 0.33 cc/g and an overall pore volume in the range of 0.28 to 0.40 cc/g.

7. The catalyst of claim 2 having an increased hydrogenation activity as compared to a copper silicate catalyst having no alkali metal component or alkaline earth metal component as used in hydrogenation reactions.

8. The catalyst of claim 1, wherein the clay material comprises an attapulgite, a sepiolite, a serpentine, a kaolinite, a calcium montmorillonite, or mixtures thereof.

9. A catalyst for hydrogenation formed from a blend consisting essentially of copper oxide, sodium hydroxide, silica sol, and a clay component, which are processed together to form a catalyst that has a mesopore volume of 50-90 by weight % of an overall pore volume.

10. The catalyst of claim 9 in extruded form having a mesopore volume in the range of 0.29 to 0.33 cc/g and an overall pore volume in the range of 0.35 to 0.40 cc/g.

11. A method of making a catalyst for hydrogenation comprising:
mixing at least one metal component comprising a metal selected from the group consisting of copper, manganese, zinc, nickel, cobalt, and iron; and an inorganic matrix component based on at least a silica sol component and a clay material to form a dry mixture;
adding a solution containing an alkali metal component to the dry mixture to form a blend; and
forming the catalyst which has a mesopore volume in the range of 50-90 by weight % of an overall pore volume.

12. The method of claim 11, wherein the metal comprises copper and the blend comprises:
an amount of the copper component in the range of 30 to 85% by weight of the blend;
an amount of the alkali metal component in the range of 0.5 to 5.0% by weight of the blend; and
a combined amount of the silica sol and clay material in the range of 15 to 70% by weight of the blend.

13. The method of claim 11, wherein the blend consists essentially of copper oxide, sodium hydroxide, silica sol, and clay.

14. A method for making alcohols or amines comprising:
providing a feedstock comprising a carbonyl compound or a nitro-compound;
contacting the feedstock with the catalyst of claim 1; and
yielding alcohols or amines, respectively.

15. The method of claim 14, wherein the metal of the catalyst comprises copper and the catalyst is prepared from a blend consisting essentially of:
an amount of the copper component in the range of 30 to 85% by weight of the blend;
an amount of the alkali metal component in the range of 0.5 to 5.0% by weight of the blend; and
a combined amount of the silica sol component and clay material in the range of 15 to 70% by weight of the blend.

16. The method of claim 14, wherein the catalyst is effective to convert 80% or more of acetophenone to methylphenyl carbinol under continuous stirred tank reactor (CSTR) conditions at 20.7 bar and feed rate of 150 cc-hr⁻¹ with 33 cc catalyst and temperatures up to 100° C. at steady state.

17. The method of claim 16, wherein the catalyst is effective to maintain 90% or more selectivity of acetophenone to methylphenyl carbinol for at least 250 hours.

18. The method of claim 17, wherein the catalyst is effective to maintain 97% or more selectivity of nitrobenzene to aniline under fixed bed conditions at 220° C. and 0.3 LHSV hr⁻¹ for at least 250 hours.

19. A method of converting acetophenone to methylphenyl carbinol, the method comprising contacting the catalyst of claim 1 with the acetophenone.

20. A method of converting nitrobenzene to aniline, the method comprising contacting the catalyst of claim 1 with the nitrobenzene.
